(19) United States
(12) Patent Application Publication
(10) Pub. No.: US 2016/0089078 A1
(43) Pub. Date: Mar. 31, 2016

(54) DEVICE AND METHOD FOR SLEEP MONITORING

(71) Applicant: SKY LIGHT ELECTRONIC (SHENZHEN) LIMITED CORPORATION, Shenzhen (CN)

(72) Inventors: Kaimeng Du, Shenzhen (CN); Tsuen Wai David Fu, Shenzhen (CN)

(73) Assignee: SKY LIGHT ELECTRONIC (SHENZHEN) LIMITED CORPORATION

(21) Appl. No.: 14/568,166

(22) Filed: Dec. 12, 2014

(30) Foreign Application Priority Data
Sep. 26, 2014 (CN) 201410505254.9

(51) Int. Cl. A61B 5/00; A61B 5/0402; A61B 5/113; A61B 8/02; A61B 5/0295; A61B 5/0205; A61B 5/145

(52) U.S. Cl. CPC A61B 5/4812; A61B 5/0205; A61B 5/0402; A61B 5/14542; A61B 8/02; A61B 5/0295; A61B 5/113; A61B 5/7278; A61B 5/7445; A61B 5/7475; A61B 5/7455; A61B 5/7405; A61B 5/0017; A61B 5/0245

(57) ABSTRACT

Disclosed is a device for sleep monitoring, which includes a detecting module, a processing module electrically connected with the detecting module and an output module electrically connected with the processing module. The detecting module is used for collecting physiological feature signals which represent sleep state of user, the physiological feature signals include one or more than one of heart rate feature signals which represent the heart rate of user and respiration feature signals which represent the respiration of user; the processing module is used for processing the physiological feature signals from the detecting module so as to obtain evaluation results of sleep of user; and the output module is used for outputting the evaluation results of sleep from the processing module. By collecting the physiological feature signals which represent the heart rate and/or respiration of user during sleep, the sleep quality of user can be evaluated accurately.

### CROSS-REFERENCE TO RELATED APPLICATIONS

This present invention claims the benefit of Chinese Patent Application No. CN201410505254.9, filed on Sep. 26, 2014; the contents of which are hereby incorporated by reference.

### FIELD OF THE INVENTION

The present invention relates to monitoring technology and, in particular, it concerns device and method for sleep monitoring.

### BACKGROUND OF THE INVENTION

The existing products for sleep monitoring fall into two broad categories: for household use and for medical use.

The products for sleep monitoring for household use are generally designed to be wearable and mostly designed based on accelerometers. In such a product, accelerometer is used to obtain motion data of user during sleep and micro-controller is used to analyze the data so as to evaluate the sleep state of user. However, the accelerometer only can be used to record body movements of user, which have no direct relevance with the sleep state, thus, it will bring two following false impressions which lead to inaccurate evaluation results of sleep quality: the first impression is that when the wearer lies awake in bed but his/her body does not move, the product for sleep monitoring will make an erroneous judgment that the wearer is in a sleep state because of no movement of the accelerometer; and the second impression is that when the wearer is asleep but his/her body moves because of mosquito bites or itchy skin and so on, the product for sleep monitoring will make an erroneous judgment that the wearer is not in a sleep state because of the movement of the accelerometer.

The products for sleep monitoring for medical use are provided for evaluating sleep quality of sleeper by measuring brain wave. Because brain wave is golden rule for determining sleep state, the existing medical device for detecting brain wave can be used to evaluate the sleep quality of user accurately. However, because the brain wave signal is very weak, the equipment used for recording the brain wave needs high-precision, which leads to that the existing medical product for sleep monitoring has high product cost, big volume, and it is inconvenient to use. Thus, at present, it is only used for health service such as in the hospital, in the clinic and so on, and it is not suitable for use at home. Furthermore, when using such a product to make sleep monitoring, many electrodes need to be placed on the head of user, which will bring discomfort and affect sleep.

Thus, it is necessary to provide a device for sleep monitoring with advantages of simple structure, convenience to use and accurate measuring, and a method thereof.

### SUMMARY OF THE INVENTION

One object of the present invention is to provide a device for sleep monitoring with advantages of simple structure, convenience to use and accurate measuring.

Another object of the present invention is to provide a method for sleep monitoring with advantages of convenience to operate and accurate measuring.

To achieve the above object, there is provided a device for sleep monitoring, which includes a detecting module, a processing module electrically connected with the detecting module and an output module electrically connected with the processing module. The detecting module is used for collecting physiological feature signals which represent sleep state of user; the processing module is used for processing the physiological feature signals from the detecting module so as to obtain evaluation results of sleep of user; and the output module is used for outputting the evaluation results of sleep from the processing module. Wherein the detecting module includes a heart rate sensor and/or a respiration sensor, the heart rate sensor being provided for collecting heart rate feature signals which represent the heart rate of user and the respiration sensor being provided for collecting respiration feature signals which represent the respiration of user.

Preferably, the heart rate sensor includes one or more than one of electrocardiograph sensor, blood oxygen saturate sensor, ultrasonic sensor, volume measurement sensor and radio frequency sensor; and the respiration sensor includes one or more than one of displacement sensor, strain sensor and volume measurement sensor.

Preferably, the detecting module further includes a motion sensor, which is provided for collecting motion feature signals which represent the action state of user.

Preferably, the processing module includes: an extraction unit, provided for extracting the physiological feature signals from the detecting module in real time; a micro-controller unit, electrically connected with the extraction unit and provided for processing the physiological feature signals by using weighted average method so as to obtain evaluation results of sleep; and a storage unit, electrically connected with the micro-controller unit and provided for storing the physiological feature signals and evaluation results of sleep.

Preferably, the output module includes one or more than one of output interface, display screen, vibrator, sounder and LED indicator light.

Preferably, the output interface includes wired interface, which includes one or more than one of USB interface, UART interface and RS-232 serial interface.

Preferably, the output interface includes wireless interface, which includes one or more than one of infrared module, Bluetooth module, 2.4G radio frequency module, 5G radio frequency module and WIFI module.

To achieve the above object, there is further provided a method for sleep monitoring, which includes following steps: (a) collecting physiological feature signals which represent sleep state of user, said physiological feature signals comprising heart rate feature signals which represent the heart rate of user and/or respiration feature signals which represent the respiration of user; (b) processing the physiological feature signals so as to obtain evaluation results of sleep of user; and (c) outputting the evaluation results of sleep.

Preferably, the physiological feature signals collected by the step (a) further includes motion feature signals which represent the action state of user.

Preferably, the step (b) includes a step of processing the physiological feature signals by using weighted average method so as to obtain evaluation results of sleep.

Compared with the prior art, the present invention has beneficial effects as follows: the physiological feature signals which represent sleep state of user, such as heart rate feature signals which represent the heart rate of user and/or respiration feature signals which represent the respiration of user, can be collected by a detecting module and the physiological feature signals can be further processed and analyzed so as to obtain evaluation results of sleep. Because the physiological changes of human in a sleep state can be directly reflected by the changes of heart rate and/or respiration, the sleep state and sleep quality of user can be evaluated more accurately by using the device for sleep monitoring of the present invention than existing method for collecting data of sleeping posture of user. And compared with the existing method for detecting brain wave, the device for sleep monitoring of the present invention has advantages of simple structure, convenience to use and lower cost, and it can be designed to be wearable.

The present invention will become clearer by means of the following description combining the accompanying drawings, which are used to illustrate embodiments of the present invention.

### BRIEF DESCRIPTION OF THE DRAWINGS

FIG. 1 is a block diagram of a device for sleep monitoring according to first embodiment of the present invention;

FIG. 2 is a detailed block diagram of the device for sleep monitoring shown in FIG. 1;

FIG. 3 is a block diagram of a device for sleep monitoring according to second embodiment of the present invention;

FIG. 4 is a block diagram of a device for sleep monitoring according to third embodiment of the present invention;

FIG. 5 is a block diagram of a device for sleep monitoring according to fourth embodiment of the present invention; and

FIG. 6 a flow chart of a method for sleep monitoring according to an embodiment of the present invention.

### DETAILED DESCRIPTION OF ILLUSTRATED EMBODIMENTS

The technical solutions of embodiments will be clearly and completely described as follows by combining the figures of the embodiments of the present invention, and similar reference numbers in the figures represent similar components. Obviously, the embodiments described as follows are merely parts of embodiments of the present invention, but not the all. Based on the embodiments of the present invention, other embodiments created by one of ordinary skill in the art without creative work, all belong to the scope of protection of the present invention.

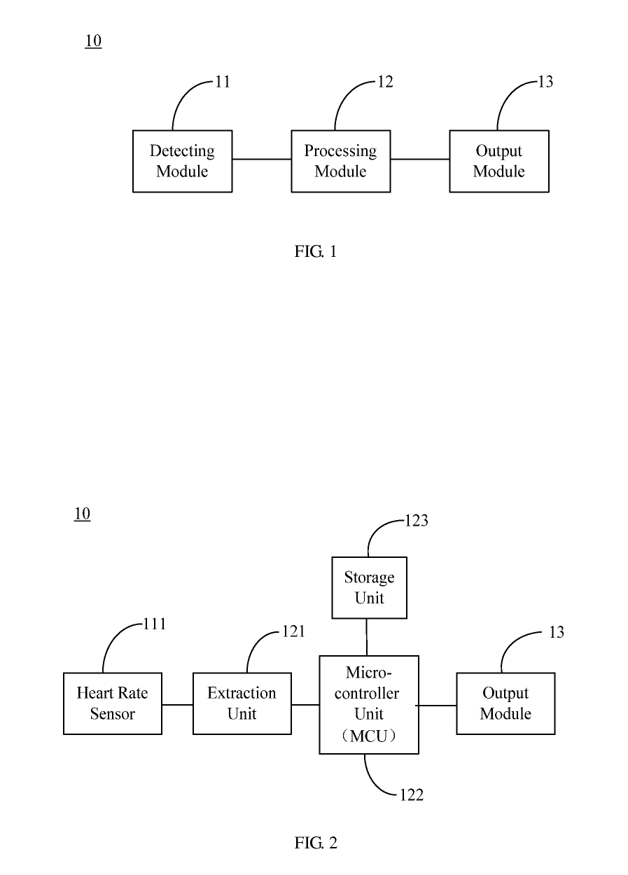

FIG. 1 and FIG. 2 illustrate a device for sleep monitoring according to first embodiment of the present invention.

Referring to FIG. 1, the device 10 for sleep monitoring of the present invention includes detecting module 11, processing module 12 and output module 13. The detecting module 11 is used for collecting physiological feature signals which represent sleep state of user; the processing module 12 is electrically connected with the detecting module 11 and used for processing the physiological feature signals from the detecting module 11 so as to obtain evaluation results of sleep of user; and the output module 13 is electrically connected with the processing module and used for outputting the evaluation results of sleep from the processing module 12. The device 10 for sleep monitoring is simple in structure and can be designed to be a wearable structure. When using, the user just needs to wear it on wrist or ankle and then the sleep monitoring will begin.

Referring to FIG. 2, in this embodiment, the detecting module 11 includes heart rate sensor 111 and the heart rate sensor 111 is used for collecting heart rate feature signals. The heart rate feature signals represent the signals which represent the heart rate of user. The heart rate sensor 111 can be achieved by one or more than one of electrocardiograph sensor, blood oxygen saturate sensor, ultrasonic sensor, volume measurement sensor and radio frequency sensor. In this embodiment, the processing module 12 includes extraction unit 121, micro-controller unit (MCU) 122 and storage unit 123. Particularly, the extraction unit 121 is electrically connected with the heart rate sensor 111 and is used for extracting the heart rate feature signals from the heart rate sensor 111 in real time; the micro-controller unit 122 is electrically connected with the extraction unit 121 and storage unit 123, and it is used for storing the real-time heart rate feature signals into the storage unit 123 and analyzing the signals so as to obtain evaluation results of sleep. The micro-controller unit 122 is further electrically connected with the output module 13 so as to output the results of monitoring. In this embodiment, the output module 13 includes one or more than one of output interface, display screen, vibrator, sounder and LED indicator light.

When making sleep monitoring by using the device 10 of this embodiment, the heart rate of user will be detected by the heart rate sensor 111 in real time, so as to get heart rate feature signals. Under the control of micro-controller unit 122, the heart rate feature signals will be extracted and amplified by the extraction unit 121 and then be stored in the storage unit 123. When the sleep is ended, the heart rate feature signals stored in the storage unit 123 will be read and then be calculated and analyzed by the micro-controller unit 122, so as to get evaluation results of sleep for evaluating the sleep quality of user. The data (including heart rate feature signals and evaluation results of sleep) stored in the storage unit 123 will be output by output module 13 so as to inform user in some way, for example, emitting a beep or producing vibration or lighting indicator light to prompt the user and showing user the specific results of monitoring via display screen.

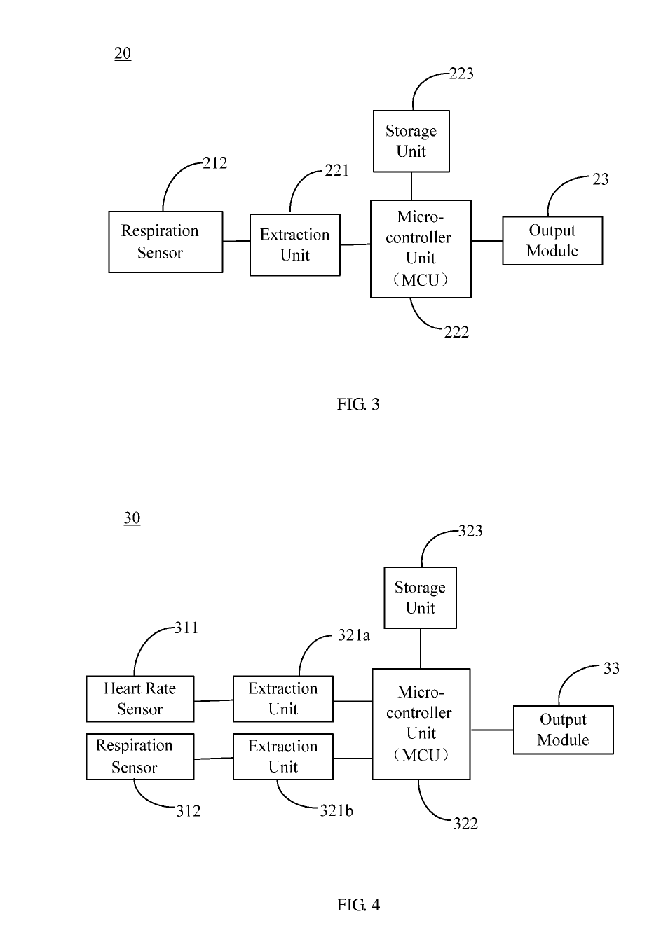

FIG. 3 illustrates a device for sleep monitoring according to second embodiment of the present invention.

Referring to FIG. 3, the main difference between the device 20 of second embodiment and the first embodiment is: the heart rate sensor 111 is replaced with respiration sensor 212 and the output module 23 is achieved by output interface. Particularly, the respiration sensor 212 is provided for collecting respiration feature signals and the respiration feature signals represent the signals which represent the respiration of user. The respiration sensor 212 can be achieved by one or more than one of displacement sensor, strain sensor and volume measurement sensor. The output interface can be wired interface or wireless interface, the wired interface being achieved by one or more than one of USB interface, UART interface and RS-232 serial interface and the wireless interface being achieved by one or more than one of infrared module, Bluetooth module, 2.4G radio frequency module, 5G radio frequency module and WIFI module.

When making sleep monitoring by using the device 20 of this embodiment, the respiration of user will be detected by the respiration sensor 212 in real time, so as to get respiration feature signals. Under the control of micro-controller unit 222, the respiration feature signals will be extracted and amplified by the extraction unit 221 and then be stored in the storage unit 223. When the sleep is ended, the respiration feature signals stored in the storage unit 223 will be read and then be calculated and analyzed by the micro-controller unit 222, so as to get evaluation results of sleep for evaluating the sleep quality of user. Finally, by means of output interface, the data (including respiration feature signals and evaluation results of sleep) stored in the storage unit 223 can be transmitted to external electronic equipment, such as computer, mobile phone or other electronic equipment, so as to be displayed or be further analyzed, and also can be uploaded to a database or a server so that the user can obtain more comprehensive analysis and guidance about sleep.

FIG. 4 illustrates a device for sleep monitoring according to third embodiment of the present invention.

Referring to FIG. 4, the device 30 of third embodiment combines the advantages of the device 10 of first embodiment and the device 20 of second embodiment. In this embodiment, the detecting module includes heart rate sensor 311 and respiration sensor 312; the output module 33 includes output interface, display screen and one or more than one of vibrator, sounder and LED indicator light; and the processing module 12 includes two extraction units 321a and 321b for extracting the signals from heart rate sensor 311 and the signals from respiration sensor, respectively.

When making sleep monitoring by using the device 30 of this embodiment, the heart rate of user will be detected by the heart rate sensor 311 in real time so as to get heart rate feature signals, at the same time, the respiration of user will be detected by the respiration sensor 312 in real time so as to get respiration feature signals. Under the control of micro-controller unit 322, the heart rate feature signals will be extracted and amplified by the extraction unit 321a and then be stored in the storage unit 323; and the respiration feature signals will be extracted and amplified by the extraction unit 321b and then be stored in the storage unit 323. When the sleep is ended, the heart rate feature signals and respiration feature signals stored in the storage unit 323 will be read and then be calculated and analyzed by the micro-controller unit 322, so as to get evaluation results of sleep for evaluating the sleep quality of user. Finally, The data (including heart rate feature signals, respiration feature signals and evaluation results of sleep) stored in the storage unit 323 will be output by output module 33 so as to inform user in some way, for example, emitting a beep or producing vibration or lighting indicator light to prompt the user and showing user the specific results of monitoring via display screen. At the same time, by means of output interface, the data can be transmitted to external electronic equipment, such as computer, mobile phone or other electronic equipment, so as to be displayed or be further analyzed, and also can be uploaded to a database or a server so that the user can obtain more comprehensive analysis and guidance about sleep.

FIG. 5 illustrates a device for sleep monitoring according to the fourth embodiment of the present invention.

Referring to FIG. 5, the main difference between the device 40 of the fourth embodiment and the device 30 of the third embodiment is: the detecting module of this embodiment not only includes a heart rate sensor 411 and a respiration sensor 412 but also a motion sensor 413. Accordingly, there are provided three extraction units 421a, 421b, and 421c, which are used for extracting the signals from heart rate sensor 411, respiration sensor 412 and motion sensor 413, respectively. Particularly, the motion sensor 413 is used for collecting motion feature signals, and the motion feature signals represent the signals which represent the action state of user. In this embodiment, the motion sensor 413 can be achieved by linear accelerometer, angular accelerometer or other sensors which can detect the movement of an object.

When making sleep monitoring by using the device 40 of this embodiment, the heart rate of user will be detected by the heart rate sensor 411 in real time so as to get heart rate feature signals; the respiration of user will be detected by the respiration sensor 412 in real time so as to get respiration feature signals; at the same time, the action state of user will be detected by the motion sensor 413 in real time so as to get motion feature signals. Under the control of micro-controller unit 422, the heart rate feature signals, respiration feature signals and motion feature signals will be extracted and amplified by the extraction units 421a, 421b and 421c, respectively, and then be stored in the storage unit 423. When the sleep is ended, the heart rate feature signals, respiration feature signals and motion feature signals all stored in the storage unit 423 will be read and then be calculated and analyzed by the micro-controller unit 422, so as to get evaluation results of sleep for evaluating the sleep quality of user. Finally, The data (including heart rate feature signals, respiration feature signals, motion feature signals and evaluation results of sleep) stored in the storage unit 423 will be output by output module 43 so as to inform user in some way, for example, emitting a beep or producing vibration or lighting indicator light to prompt the user and showing user the specific results of monitoring via display screen. At the same time, by means of output interface, the data can be transmitted to external electronic equipment, such as computer, mobile phone or other electronic equipment, so as to be displayed or be further analyzed, and also can be uploaded to a database or a server so that the user can obtain more comprehensive analysis and guidance about sleep.

In all the above-mentioned embodiments, each of the micro-controlled units 122, 222, 322 and 422 is capable of processing one or more than one kinds of physiological feature signals of the heart rate feature signals, respiration feature signals and motion feature signals by using weighted average method and then comparing and analyzing the processed data, thereby obtaining monitoring results. Particularly, the data can be processed in the following way:

Firstly, determine the sleep evaluation index according to following formula: sleep evaluation index=(a1*heart rate+a2*respiration rate+a3*motion frequency)+(b1*rate of heartbeat change+b2* rate of respiration change+b3* rate of motion change), wherein the data about heart rate, respiration rate, motion frequency, rate of heartbeat change, rate of respiration change and rate of motion change can be determined by the collected physiological feature signals, aᵢ and bᵢ are weighting coefficients, the values of which can be set according to the specific requirements.

Secondly, compare the sleep evaluation index with a preset threshold cᵢ, when the sleep evaluation index≦c₁, it means that the evaluation result of sleep is good; when the sleep evaluation index≧c₁ and the sleep evaluation index≦c₂, it means that the evaluation result of sleep is middle; and when the sleep evaluation index≧c₂, it means that the evaluation result of sleep is poor. Wherein, the value of cᵢ can be set according to the specific requirements.

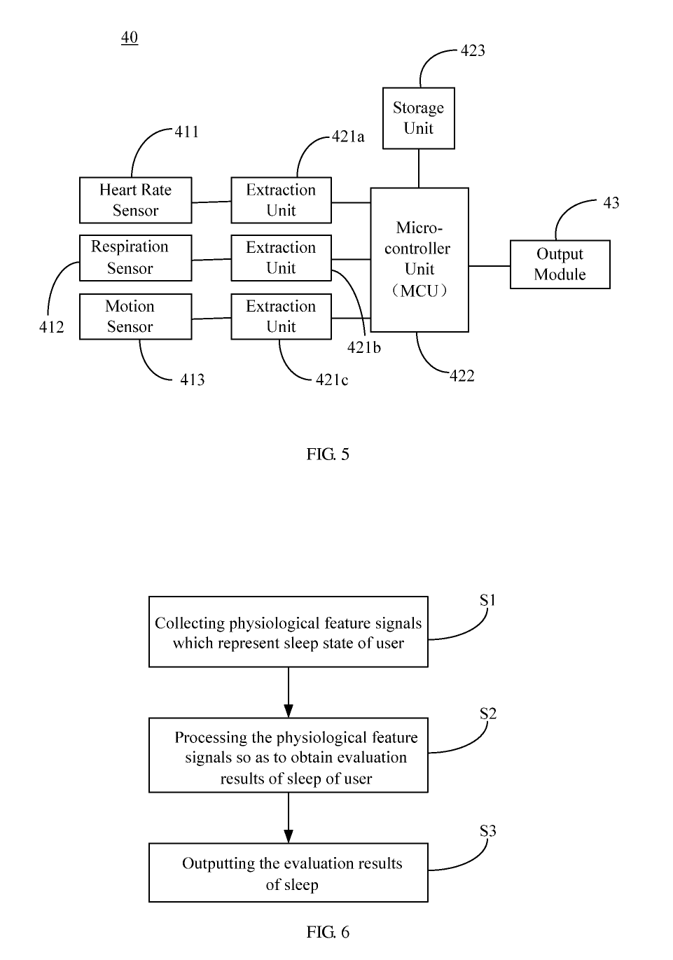

Additionally, there is provided a method for sleep monitoring. FIG. 6 shows a flow chart of the method for sleep monitoring of the present invention. The method includes following steps: firstly, collecting physiological feature signals which represent sleep state of user, the physiological feature signals including heart rate feature signals which represent the heart rate of user and/or respiration feature signals which represent the respiration of user (step S1); secondly, processing the physiological feature signals so as to obtain evaluation results of sleep of user (step S2); finally, outputting the evaluation results of sleep (step S3).

The method for sleep monitoring achieved by the device 40 for sleep monitoring according to the fourth embodiment will be illustrated as follows. Firstly, the heart rate of user will be detected by the heart rate sensor 411 in real time so as to get heart rate feature signals; the respiration of user will be detected by the respiration sensor 412 in real time so as to get respiration feature signals; at the same time, the action state of user will be detected by the motion sensor 413 in real time so as to get motion feature signals. Under the control of micro-controller unit 422, the heart rate feature signals, respiration feature signals and motion feature signals will be extracted and amplified by the extraction units 421a, 421b and 421c, respectively, and then will be stored in the storage unit 423. When the sleep is ended, the heart rate feature signals, respiration feature signals and motion feature signals all stored in the storage unit 423 will be read and then be calculated and analyzed by the micro-controller unit 422, so as to get evaluation results of sleep for evaluating the sleep quality of user. Finally, The data (including heart rate feature signals, respiration feature signals, motion feature signals and evaluation results of sleep) stored in the storage unit 423 will be output by output module 43 so as to inform user in some way, for example, emitting a beep or producing vibration or lighting indicator light to prompt the user and showing user the specific results of monitoring via display screen. At the same time, by means of output interface, the data can be transmitted to external electronic equipment, such as computer, mobile phone or other electronic equipment, so as to be displayed or be further analyzed, and also can be uploaded to a database or a server so that the user can obtain more comprehensive analysis and guidance about sleep.

According to the present invention, the physiological feature signals which represent sleep state of user, such as heart rate feature signals which represent the heart rate of user and/or respiration feature signals which represent the respiration of user, can be collected by a detecting module and the physiological feature signals can be further processed and analyzed so as to obtain evaluation results of sleep. Because the physiological changes of human in a sleep state can be directly reflected by the changes of heart rate and/or respiration, the sleep state and sleep quality of user can be evaluated more accurately by using the device for sleep monitoring of the present invention than existing method for collecting data of sleeping posture of user. And compared with the existing method for detecting brain wave, the device for sleep monitoring of the present invention has advantages of simple structure, convenience to use and lower cost, and it can be designed to be wearable.

Understandably, according to the present invention, the detecting module can be composed of heart rate sensor(s) and motion sensor(s) or be composed of motion sensor(s) and respiration sensor(s). And the sensors provided in the present invention can be wired sensors, wireless sensors or built-in sensors.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention.

What is claimed is:

1. A device for sleep monitoring, comprising:
a detecting module, provided for collecting physiological feature signals which represent sleep state of user;
a processing module, electrically connected with the detecting module and provided for processing the physiological feature signals from the detecting module so as to obtain evaluation results of sleep of user; and
an output module, electrically connected with the processing module and provided for outputting the evaluation results of sleep from the processing module;
wherein the detecting module comprises a heart rate sensor and/or a respiration sensor, the heart rate sensor being provided for collecting heart rate feature signals which represent the heart rate of user and the respiration sensor being provided for collecting respiration feature signals which represent the respiration of user.

2. The device for sleep monitoring according to claim 1, wherein the heart rate sensor comprises one or more than one of electrocardiograph sensor, blood oxygen saturate sensor, ultrasonic sensor, volume measurement sensor and radio frequency sensor; and the respiration sensor comprises one or more than one of displacement sensor, strain sensor and volume measurement sensor.

3. The device for sleep monitoring according to claim 1, wherein the detecting module further comprises a motion sensor, which is provided for collecting motion feature signals which represent the action state of user.

4. The device for sleep monitoring according to claim 1, wherein the processing module comprises:
an extraction unit, provided for extracting the physiological feature signals from the detecting module in real time;
a micro-controller unit, electrically connected with the extraction unit and provided for processing the physiological feature signals by using weighted average method so as to obtain evaluation results of sleep; and
a storage unit, electrically connected with the micro-controller unit and provided for storing the physiological feature signals and evaluation results of sleep.

5. The device for sleep monitoring according to claim 2, wherein the processing module comprises:
an extraction unit, provided for extracting the physiological feature signals from the detecting module in real time;
a micro-controller unit, electrically connected with the extraction unit and provided for processing the physiological feature signals by using weighted average method so as to obtain evaluation results of sleep; and
a storage unit, electrically connected with the micro-controller unit and provided for storing the physiological feature signals and evaluation results of sleep.

6. The device for sleep monitoring according to claim 3, wherein the processing module comprises:
an extraction unit, provided for extracting the physiological feature signals from the detecting module in real time;
a micro-controller unit, electrically connected with the extraction unit and provided for processing the physiological feature signals by using weighted average method so as to obtain evaluation results of sleep; and
a storage unit, electrically connected with the micro-controller unit and provided for storing the physiological feature signals and evaluation results of sleep.

7. The device for sleep monitoring according to claim 1, wherein said output module comprises one or more than one of output interface, display screen, vibrator, sounder and LED indicator light.

8. The device for sleep monitoring according to claim 7, wherein said output interface comprises wired interface, which comprises one or more than one of USB interface, UART interface and RS-232 serial interface.

9. The device for sleep monitoring according to claim 7, wherein said output interface comprises wireless interface, which comprises one or more than one of infrared module, Bluetooth module, 2.4G radio frequency module, 5G radio frequency module and WIFI module.

10. A method for sleep monitoring, comprising following steps:
(a) collecting physiological feature signals which represent sleep state of user, said physiological feature signals comprising heart rate feature signals which represent the heart rate of user and/or respiration feature signals which represent the respiration of user;
(b) processing the physiological feature signals so as to obtain evaluation results of sleep of user; and
(c) outputting the evaluation results of sleep.

11. The method for sleep monitoring according to claim 10, wherein the physiological feature signals collected by the step (a) further comprises motion feature signals which represent the action state of user.

12. The method for sleep monitoring according to claim 10, wherein the step (b) comprises a step of processing the physiological feature signals by using weighted average method so as to obtain evaluation results of sleep.

13. The method for sleep monitoring according to claim 11, wherein the step (b) comprises a step of processing the physiological feature signals by using weighted average method so as to obtain evaluation results of sleep.
